(19) United States
(12) Patent Application Publication
(10) Pub. No.: US 2015/0238083 A1
(43) Pub. Date: Aug. 27, 2015

(54) METHOD AND SYSTEM FOR OPTICALLY INVESTIGATING A TISSUE OF A SUBJECT

(71) Applicant: VALORISATION-RECHERCHE, LIMITED PARTNERSHIP, Montreal (CA)

(72) Inventors: Jocelyn Faubert, Montreal (CA); Claudine Habak, Saint-Laurent (CA); Rafael Doti, Montreal (CA); Jesus-Eduardo Lugo-Arce, Laval (CA)

(21) Appl. No.: 14/420,796

(22) PCT Filed: Aug. 9, 2013
(86) PCT No.: PCT/CA2013/000711

§ 371 Date: Feb. 10, 2015

Related U.S. Application Data
(60) Provisional application No. 61/681,968, filed on Aug. 10, 2012.

(51) Int. Cl. A61B 5/00

(52) U.S. Cl. CPC A61B 5/0059; A61B 2562/0238; A61B 2562/04

(57) ABSTRACT

There is described a probe device for optically investigating a tissue of a subject, comprising: a first probe element, a second probe element, and a third probe element each to be positioned at a respective vertex of a triangle for sensing the tissue, the first probe element each comprising a first light source for emitting light having a first wavelength, the second probe element each comprising a second light source for emitting light having a second wavelength and a first photodetector for detecting light having the first wavelength and scattered by the tissue, and the third probe element comprising a second photodetector for detecting light having the first and second wavelengths and scattered by the tissue.

### CROSS-REFERENCE TO RELATED APPLICATIONS

This application claims priority of US Provisional Patent Application having Ser. No. 61/681,968, which was filed on Aug. 10, 2012 and is entitled “Method and system for assessing a stimulus property perceived by a subject”, the specification of which is hereby incorporated by reference.

### TECHNICAL FIELD

The present invention relates to the field of methods and systems for investigating tissues of a subject, and more particularly to optical methods and systems for investigating tissues.

### BACKGROUND

Optical non-invasive systems are used for investigating tissues of subjects. Some optical systems comprise light emitters and light detectors positioned at different locations of the subject for sensing the tissue. When an emitter emits light, the light is scattered by the tissue and the scattered light is detected by the detectors surrounding the emitter, which allows investigating the tissue regions comprised between the emitter and each detector.

For example, such optical systems can be used for investigating a human brain in order to determine a brain activity. In some instances, it may be of importance to sense the brain regions having the greatest activity. However, a great activity brain region may be located between two adjacent detectors and therefore cannot be investigated. Therefore, the position of the emitters and/or detectors has to be changed until the great activity brain region is comprised between an emitter and a detector, which is a time-consuming process.

Therefore, there is a need for an improved method and system for optically investigating a tissue of a subject.

### SUMMARY

According to a first broad aspect, there is provided a system for optically investigating a tissue of a subject, comprising: a first probe element, a second probe element, and a third probe element each adapted to be positioned at a respective vertex of a triangle for sensing a given region of a tissue, the first probe element comprising a first light source for emitting light having a first wavelength, the second probe element comprising a second light source for emitting light having a second wavelength and a first photodetector for detecting light having the first wavelength and being scattered by the tissue, and the third probe element comprising a second photodetector for detecting light having the first and second wavelengths and being scattered by the tissue; and a control unit operatively connected to at least the first and second probe elements, the control unit being adapted to operate the second light source for sensing a first region comprised between the second and third probe elements, and operate the first light source for sensing a second and a third region respectively comprised between the first probe element and the second and third probe elements.

In one embodiment, the system further comprises a measurement collecting unit connected to the first and second photodetectors for receiving therefrom signals indicative of scattered light energies measured by the first and second photodetectors.

In one embodiment, the control unit is adapted to selectively operate the first and second light sources for selectively sensing the second and third regions, and the first region, respectively.

In another embodiment, the control unit is adapted to concurrently operate the first and second light source.

In one embodiment, the second probe element comprises an optical transceiver.

In one embodiment, the first, second, and third probe elements are each adapted to be positioned at a respective vertex of one of an isosceles triangle, a scalene triangle, and an equilateral triangle.

In one embodiment, the first wavelength emitted by the first light source and the second wavelength emitted by the second light source are substantially identical.

In one embodiment, a distance between the first light source and the first photodetector and a distance between the first light source and the second photodetector are each substantially equal to about three times a tissue depth to be investigated.

According to a second broad aspect, there is described a method for optically investigating a tissue of a subject, comprising: positioning, on the subject, a first probe element, a second probe element, and a third probe element each at a respective vertex of a triangle for sensing a given region of the tissue, the at least one first probe element each comprising a first light source for emitting light having a first wavelength, the at least one second probe element each comprising a second light source for emitting light having a second wavelength and a first photodetector for detecting light having the first wavelength and scattered by the tissue, and the at least one third probe element comprising a second photodetector for detecting light having the first and second wavelengths and scattered by the tissue; operating the second light source for sensing a first tissue region comprised between the second and third probe elements; and operating the first light source for respectively sensing a second tissue region comprised between the first probe and the second probe, and a third tissue region comprised between the first probe and the third probe.

In one embodiment, the steps of operating the first light source and operating the second light source are selectively performed for selectively sensing the second and third regions, and the first region, respectively.

In another embodiment, the steps of operating the first light source and operating the second light source are performed substantially concurrently.

In one embodiment, the second probe element comprises an optical transceiver.

In one embodiment, the positioning step comprises positioning the first probe element, the second probe element, and the third probe element each at a respective vertex of one of an isosceles triangle, a scalene triangle, and an equilateral triangle.

In one embodiment, the first wavelength emitted by the first light source and the second wavelength emitted by the second light source are substantially identical.

In one embodiment, the positioning step comprises positioning the first, second, and third probe elements so that a distance between the first light source and the first photodetector and a distance between the first light source and the second photodetector are each substantially equal to about three times a tissue depth to be investigated.

In accordance with a further broad aspect, there is provided a probe device for optically investigating a tissue of a subject, comprising: a first probe element, a second probe element, and a third probe element each to be positioned at a respective vertex of a triangle for sensing the tissue, the first probe element each comprising a first light source for emitting light having a first wavelength, the second probe element each comprising a second light source for emitting light having a second wavelength and a first photodetector for detecting light having the first wavelength and scattered by the tissue, and the third probe element comprising a second photodetector for detecting light having the first and second wavelengths and scattered by the tissue, the second light source and the first photodetector being respectively adapted for sensing a first tissue region comprised between the second and third probes, and a second and third tissue regions comprised between the first probe and the second and third probes.

In one embodiment, the first, second, and third probe elements are each removably securable to the subject at an adequate location for sensing the tissue.

In another embodiment, the probe device further comprises a frame securable to the subject.

In one embodiment, the first, second, and third probe elements are secured to the frame.

In another embodiment, the first, second, and third probe elements are remotely and optically connected to the frame.

The term “tissue” refers to a cellular organizational level intermediate between cells and a complete organism. A tissue is an ensemble of cells, not necessarily identical, but from the same origin, that together carry out a specific function. Organs are then formed by the functional grouping together of multiple tissues.

### BRIEF DESCRIPTION OF THE DRAWINGS

Further features and advantages of the present invention will become apparent from the following detailed description, taken in combination with the appended drawings, in which:

FIG. 1 schematically illustrates a probe system of the prior art comprising one light emitter and two photodetectors;

FIG. 2 schematically illustrates a probe system of the prior art comprising five light emitters and four photodetectors;

FIG. 3 schematically illustrates a probe system comprising one light emitter, a photodetector, and a transceiver, in accordance with an embodiment;

FIG. 4 is a flow chart of a method for optically investigating a tissue, in accordance with an embodiment;

FIG. 5 schematically illustrates a probe system having a hexagonal configuration, in accordance with an embodiment;

FIG. 6 schematically illustrates a probe system having a square configuration, in accordance with an embodiment;

FIG. 7 illustrates an investigation channel in a brain, in accordance with an embodiment;

FIGS. 8a-8h schematically illustrate an array of transceivers allowing scanning of different regions, in accordance with an embodiment;

FIGS. 9a-9b schematically illustrate an array of transceivers adapted to generate probe unit of different configurations, in accordance with an embodiment;

FIGS. 10a and 10b illustrates two supports for probe systems comprising an array of transceivers, in accordance with an embodiment;

FIG. 11 is a block diagram of a system for measuring brain activity of a subject following a visual stimulation, in accordance with an embodiment; and

FIG. 12 is a block diagram of a system for dynamically controlling a probe system, in accordance with an embodiment.

It will be noted that throughout the appended drawings, like features are identified by like reference numerals.

### DETAILED DESCRIPTION

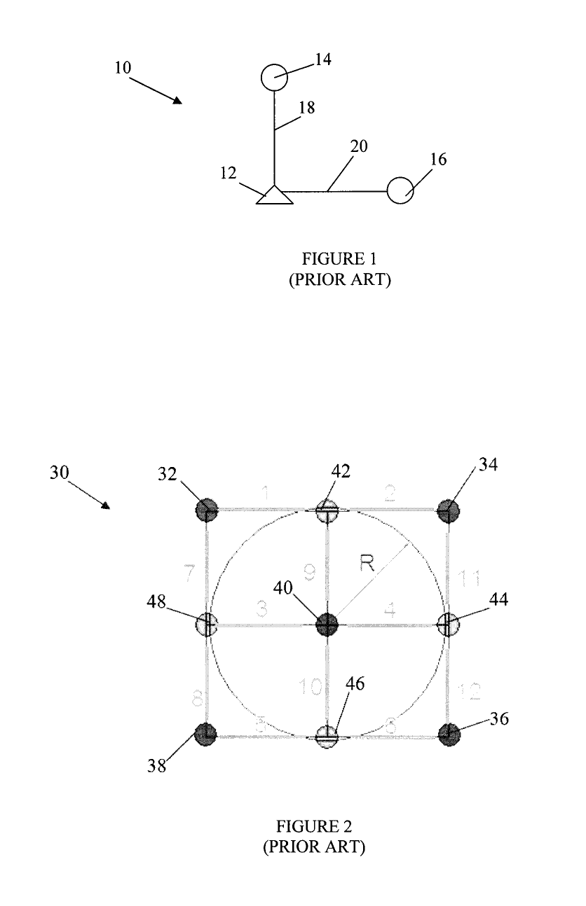

FIG. 1 schematically illustrates an exemplary probe system 10 according to the prior art. The probe system 10 comprises a light emitter or light source 12 adapted to generate light having a given wavelength, and a first and a second photodetector or light detector 14 and 16 each adapted to detect and measure light having the given wavelength.

The light emitter 12 and the photodetectors 14 and 16 are positioned on a subject, such as a human being, an animal, a mammal, or the like, for sensing a given tissue. The photodetectors are positioned substantially at equal distance from the light emitter 12. The light emitter 12 emits a pulse of light that propagates through the given tissue. A first portion of the light propagates in the tissue region located between the light emitter 12 and the photodetector 14 while a second portion of the light propagates in the tissue region located between the emitter 12 and the second photodetector 16. The two tissue regions scatter the light propagating therein and the photodetectors 12 and 14 each detect the light scattered by their respective tissue region. The photodetector 14 is said to measure the channel 18 located between the emitter 12 and the photodetector 14. Similarly, the photodetector 16 is said to measure the channel 20 located between the emitter 12 and the photodetector 16. A channel is defined as a pathway through which an optical signal is transmitted and that links a transmitting point and a receiving point. The channel is established when the transfer of that signal presents enough efficiency to permit receiving and decoding the information transmitted.

Therefore, the probe system 10 can only measure two channels. For example, the region between the two photodetectors 14 and 16 cannot be investigated since no light can propagate between the two photodetectors 14 and 16. For example, if the tissue region between two photodetectors 14 and 16 would be of interest, one should change the position of the probes. For example, after investigating the channels 18 and 20, the channel between the photodetectors 14 and 16 may be investigated by inverting the position of the emitter 12 and the photodetector 16 or 18. Changing the position of the probes is a time consuming step and increases the time required to investigate the tissue.

FIG. 2 illustrates another exemplary probe system 30 of the prior art. The probe system 30 comprises five light emitters 32-40 and four photodetectors 42-48, which are positioned according to a square pattern. Each one of the emitters 32-38 are positioned at a respective corner of the square pattern while the emitter 40 is positioned at the center of the square pattern. The four photodetectors 42-48 are each positioned at the center of a respective edge of the square pattern. Similarly to the probe system 10, the position of some of the emitters 32-40 and that of some of the photodetectors 42-48 must be inverted to investigate some regions such as the regions between the center and the corners of the square pattern.

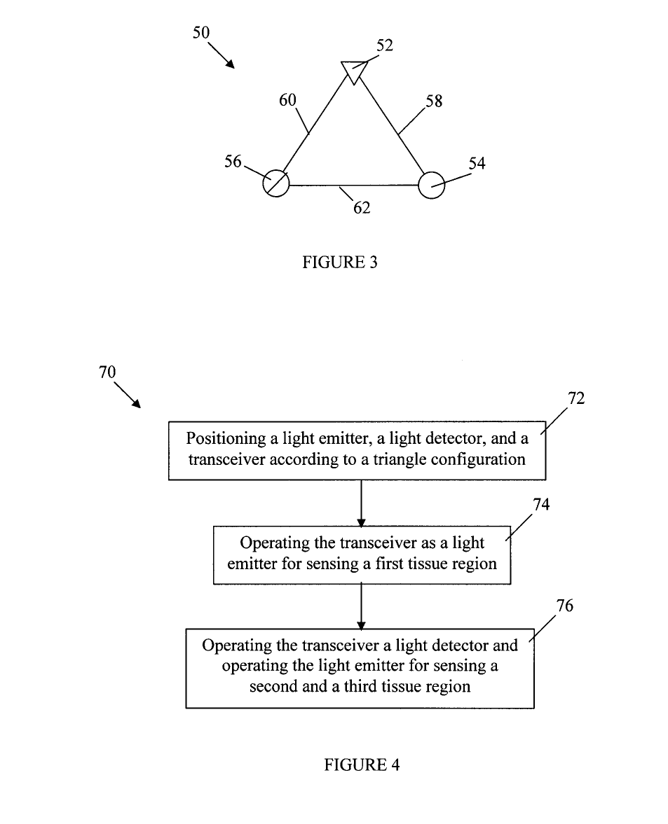

FIG. 3 illustrates one embodiment of a unitary probe system 50 which comprises a first probe, i.e. a light emitter 52, a second probe, i.e. a light detector or photodetector 54, and a third probe, i.e. an optical transceiver 56, each to be positioned at a different location on the subject for sensing a given tissue. The light emitter 52 is adapted to emit light having a first wavelength that the light detector 54 is adapted to detect and measure the energy, i.e. the amplitude, intensity, or power.

The optical transceiver 56 comprises a light emitter for emitting light having a second wavelength and a photodetector adapted to detect light having the first wavelength and measure its energy. The light detector 54 is further adapted to detect light having the second wavelength and measure its energy.

When the probe system 50 is connected to a control unit (not shown), the assembly forms a system for optically investigating a tissue of a subject. The control unit is adapted to operate the light emitter 52, the photodetector 54, and the transceiver 56. The system for optically investigating the tissue operates according to the method illustrated in FIG. 4. At step 72, the light emitter 52, the light detector 54, and the optical transceiver 56 are each positioned on the subject of which a tissue is to be investigated at an adequate position relative to the tissue. Furthermore, the light emitter 52, the light detector 54, and the optical transceiver 56 are positioned according to a triangle configuration, i.e. they are each positioned at a respective vertex of a triangle, as illustrated in FIG. 3. At step 74, the transceiver 56 is operated as a light emitter so that light having the second wavelength is emitted and scattered by the tissue. At least some of the scattered light is detected by the light detector 54. At step 76, the transceiver 56 is operated as a light detector adapted to detect light having the first wavelength and the light emitter 52 is operated to emit light having the first wavelength. The tissue scatters the light emitted by the light emitter 52 and at least some of the scattered light is detected by the light detector 54 and the transceiver 56.

It should be understood that the control unit may be further adapted to control characteristics of the light emitter 52 and the light emitter of the transceiver 54 such as the intensity or power or energy of the light emitted, the frequency and time duration of pulses of light emitted, the wavelength of the emitted light, and/or the like.

In one embodiment, steps 74 and 76 are performed successively. For example, step 74 may be performed previously to step 76. In another embodiment, step 76 is performed previously to step 74.

In another embodiment, steps 74 and 76 are performed substantially concurrently together.

When the unitary probe system 50 is positioned on a subject for sensing a tissue, e.g. on the forehead of a human subject for investigating the cerebral activity of the frontal cortex, three channels 58, 60, and 62 may be investigated. The channel 58 corresponds to a first tissue region, e.g. a first frontal cortex region, comprised between the light emitter 52 and the light detector 54, the channel 60 corresponds to a second tissue region, e.g. a second frontal cortex region, comprised between the light emitter 52 and the transceiver 56, and the channel 62 corresponds to a third tissue region, e.g. a third frontal cortex region, comprised between the transceiver 56 and the light detector 54.

When the light emitter 52 emits light, a portion of the emitted light propagates in the first tissue region located between the emitter 52 and the light detector 54, e.g. in the first frontal cortex region. The first tissue region scatters light and some of the scattered light is collected and measured by the light detector 54 in order to investigate the first channel 58. Furthermore, another portion of the emitted light propagates in the second tissue region located between the emitter 52 and the light detector of the transceiver 56, e.g. in the second frontal cortex region. The second tissue region scatters light and some of the scattered light is collected and measured by the light detector of the transceiver 56 in order to investigate the second channel 60.

When the light emitter of the transceiver 56 emits light, a portion of the emitted light propagates in the third tissue region located between the transceiver 56 and the light detector 54, e.g. in the third frontal cortex region. The third tissue region scatters light and some of the scattered light is collected by the photodetector 54 in order to investigate the third channel 62.

The light emitter 52, the light detector 54, and the transceiver 56 are positioned according to a triangle configuration, i.e. they are each positioned at a respective vertex of a triangle. In one embodiment, the distance between the light emitter 52 and the photodetector 54, the distance between the emitter 52 and the transceiver 56, and the distance between the transceiver 56 and the light detector 54 are all different. In another embodiment, the light emitter 52, the light detector 54, and the transceiver 56 are positioned according to an isosceles triangle configuration, i.e. they are each positioned at a respective vertex of an isosceles triangle. In this case, the distance between the light emitter 52 and the photodetector 54 may be substantially equal to the distance between the emitter 52 and the transceiver 56, but different from the distance between the transceiver 56 and the light detector 54, for example. In a further embodiment, the light emitter 52, the light detector 54, and the transceiver 56 are positioned according to an equilateral triangle configuration, i.e. they are each positioned at a respective vertex of an equilateral triangle. In this case, the distance between the light emitter 52 and the photodetector 54, the distance between the emitter 52 and the transceiver 56, and the distance between the transceiver 56 and the light detector 54 are all substantially equal. In still another embodiment, the light emitter 52, the light detector 54, and the transceiver 56 are positioned according to a scalene triangle configuration.

The unitary probe system 50 is connected to a control unit (not shown) and a measurement collecting unit (not shown). The control unit is adapted to control the operation of the light emitter 52, the light detector 54, and the transceiver 56, as described above. The measurement collecting unit is connected to the light detector 54 and the transceiver 56 for receiving signals indicative of the scattered light energy measured by the light detector 54 and the light detector of the transceiver 56. The measurement collecting unit may be further adapted to determine a characteristic of the tissue according to the received scattered light measurement. For example, the measurement collecting unit may be adapted to determine the cerebral activity associated to the channels 58-62 from the measurement of the scattered light. For example, the blood oxygenation of the frontal cortex regions may be determined in order to know the cerebral activity. In another example, the measurement collecting unit may be adapted to receive the scattered light measurements from the unitary probe system 50 and determine, therefrom, the electrical activity, the blood flow, the temperature, and/or the like.

In one embodiment, the light emitter and the light detector of the transceiver 56 operate concurrently, i.e. the light emitter may emit a light pulse while the light detector collects scattered light. In this case, the light detector of the transceiver 56 is adapted to detect a wavelength different from that emitted by the light emitter of the transceiver 56. Alternatively, the light detector of the transceiver 56 is adapted to detect the wavelength emitted by the light emitter of the transceiver 56 and any transceiver 56 crosstalk is substantially eliminated or reduced using any adequate method.

In another embodiment, the light emitter and the light detector of the transceiver 56 operates selectively. In this case, the control unit is adapted to selectively operate the light emitter and the light detector of the transceiver 56. For example, the control unit first operates the transceiver 56 as a light detector and controls the light emitter 52 to emit a light pulse in order to investigate the channels 58 and 60. Subsequently, the control unit operates the transceiver as light emitter so that the light emitter of the transceiver 56 emits a light pulse to be detected by the light detector 54 in order to investigate the channel 62. In this case, the wavelength of the light emitted by the emitter 52 and that of the light emitted by the transceiver 56 may be substantially the same. Alternatively, they may be different.

In one embodiment, the control unit is adapted to desynchronize the operation of the light emitter 52 and the light emitter of the transceiver 56 so that they do not concurrently emit light.

In one embodiment, the second wavelength emitted by the transceiver 56 is substantially equal to the first wavelength emitted by the emitter 52. In another embodiment, the first and second wavelengths are different.

In one embodiment, the light emitter 52 is part of a transceiver so that the probe system 50 comprises two transceivers and a light detector 54. In another embodiment, the light detector 54 is part of a transceiver so that the probe system 50 comprises two transceivers and a light emitter 52. In a further embodiment, the light emitter 52 and the light detector 54 are each part of a respective transceiver so that the probe system comprises three transceivers.

It should be understood that a probe system may comprise more than one light emitter 52, more than one light detector 54, and/or more than one transceiver. It should also be understood that a transceiver 56 may comprise more than one light emitter and/or more than one light detector.

In one embodiment, the light emitter 52 and/or the light emitter of the transceiver 56 are adapted to emit selectively or concurrently at least two different wavelengths.

For example, FIG. 5 illustrates one embodiment of a probe system 78 which comprises one light emitter 52, three light detectors 54, and three transceivers 56, which are positioned according to a hexagonal configuration, i.e. the light detectors 54 and the transceivers are each positioned a respective vertex of an hexagon. The person skilled in the art will appreciate that the above-described triangular configuration is respected since the hexagon comprises six triangles.

When the transceivers 56 each operate as a light detector and the light emitter 52 emits light, the probe system 78 is adapted to investigate three channels 58 and three channels 60. When the transceivers 56 operate as light emitters, the probe system 78 is adapted to investigate six channels 62.

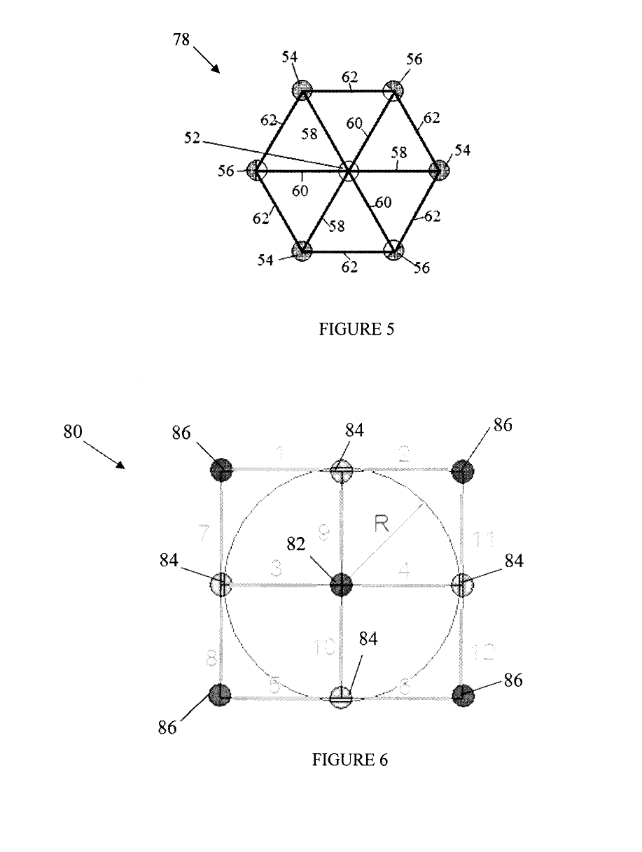

It should be understood that the at least one light emitter 52, the at least one light detector 54, and the at least one transceiver 56 may have any adequate geometrical configuration. For example, FIG. 6 illustrates one embodiment of a probe system 80 comprising a light emitter 82, four light detectors 84, and four transceivers 86, which are positioned according to a square configuration. The emitter 82 is positioned at the center of the square. The transceivers 86 are each positioned at a respective corner of the square. The light detectors 84 are each positioned at the center of a respective edge of the square.

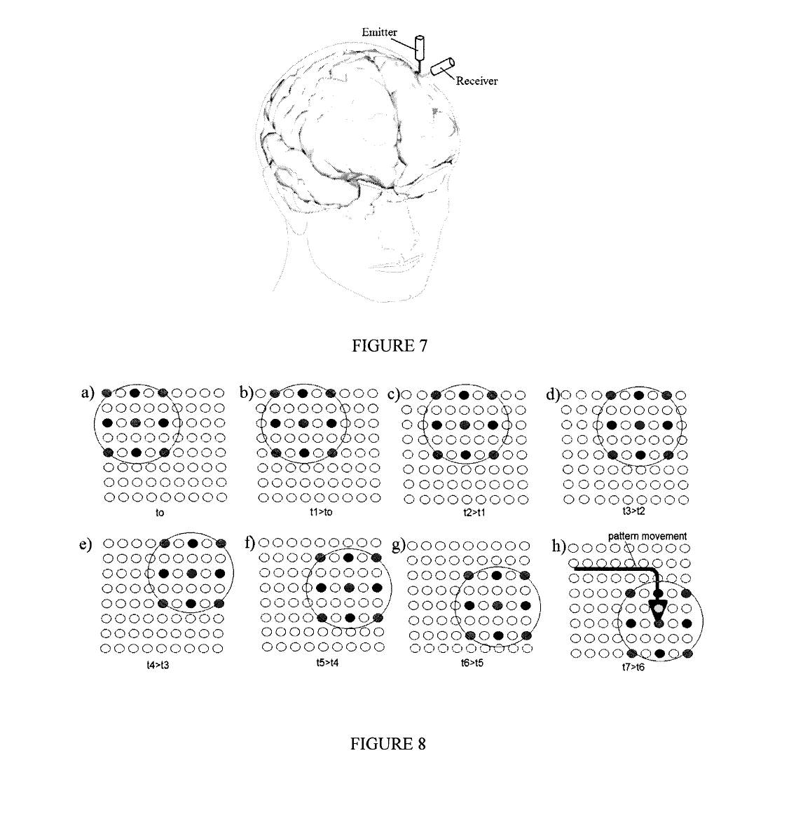

In one embodiment, the distance between the probes, i.e. the light emitter(s), the light detector(s), and the transceiver(s), is determined at least as a function of the deepness of the tissue region to be investigated. FIG. 7 illustrates a channel generated for investigating a brain region when light is emitted by a light emitter and collected by a light detector. The channel length is function of the distance between the light emitter and the light detector, the tissue optical characteristics, and the light. The distance between the light emitter and the light detector establishes the penetration depth of the light. Therefore, a particular tissue region located at a given depth may be investigated by adequately choosing the distance between a light emitter and a light detector, and different layers of tissue may be investigated by changing the distance between a light emitter and a light detector.

In an embodiment in which a human brain is to be investigated, the distance between a light emitter and a light detector is about 3 times the depth of the brain region to be investigated, when the emitted light wavelength is about 670 nm. For example, a penetration depth having a value comprised between about 8 mm and about 9 mm may be sufficient to reach the subject's forehead brain tissue, such as superficial brain tissue for example.

FIGS. 8a-8h illustrate a probe system comprising 72 transceivers positioned according to an array of 9 columns and 8 rows. Each one of the transceivers may be independently operated as a light emitter or a light receiver. The distance L between two adjacent transceivers along a row or a column is substantially equal to half of the distance corresponding to the length of a channel (2 L).

It should be understood that other configurations are possible. For example, the distance L between two following transceivers along a row or a column may be substantially equal to one quarter of the distance corresponding to the length of a channel.

As illustrated in FIG. 8a, a probe unit comprises 9 transceivers organized according to a square configuration. The transceiver positioned at the center of the square operates as a light emitter as well as the transceivers located at each corner of the square. The four transceivers each located at the center of a respective edge of the square operate as light detectors.

The control unit concurrently operates only nine transceivers, i.e. five transceivers operating as light emitters and four transceivers operating as light detectors, while the other 83 transceivers are not activated. By applying five horizontal translations to the probe unit of FIG. 8a each by a distance equal to half of the distance corresponding to the channel length, it is possible to scan a horizontal band of tissue as illustrated in FIGS. 8a-8e. Then, by applying four vertical translations the probe unit of FIG. 8e each by a distance equal to half of the distance corresponding to the channel length, it is possible to scan a vertical band of tissue as illustrated in FIGS. 8e-8h. Therefore, it is possible to scan a tissue without changing the position of the probes, i.e. changing the position of any transceiver. For example, the scan may be performed to determine the particular channels that provide the scattered light signals having the highest intensity. After this identification, only the channels presenting the highest scattered light intensity may be used for subsequent measurements.

In one embodiment, the distance R between probes is 2×about 12.7 mm=about 25.4 mm. The pattern can then adopt 5 different positions horizontally (it is able to move 5×about 2.7 mm=about 63.5 mm in the horizontal direction) and 4 different positions vertically (it is able to move 4×about 12.7 mm=about 50.8 mm in the vertical direction) covering a total area of: about 100.8 mm×about 88.9 mm, and being able to adopt 20 different positions without the need of physically changing the position of a single transceiver.

It should be understood that the pattern configuration for the active transceivers may vary and that the number of transceivers being concurrently active may vary.

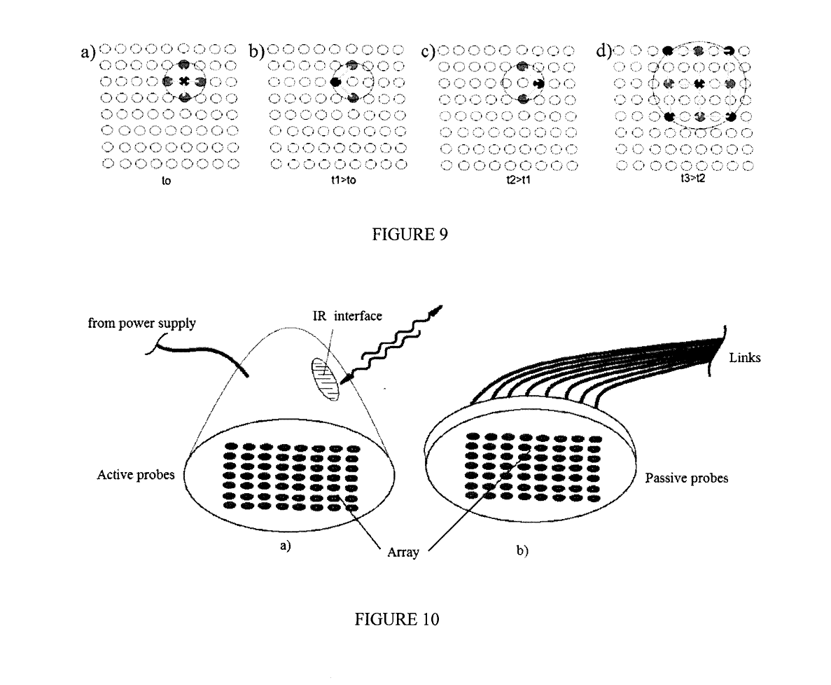

FIGS. 9a-9c illustrate different pattern configurations that may be achieved using the array of transceivers of FIG. 8a. While the geometrical configuration for a probe unit, i.e. the geometrical configurations for concurrently active transceivers, is changing, it is also possible to change the length of channels, and therefore investigate tissue regions having different depths.

In FIG. 9a, a single transceiver is operated as a light emitter while the four transceivers located at a distance L from the light emitter are operated as light detectors. In this case, it is possible to investigate four channels each corresponding to an emitter-detector distance equal to L.

In FIGS. 9b and 9c, a single transceiver is operated as a light emitter while two other transceivers are concurrently operated as light detectors. Each one of the two light detectors is located at a distance equal to (√2)L from the emitter. In this case, it is possible to investigate two channels each corresponding to an emitter-detector distance equal to (√2)L.

In FIG. 9d, five transceivers are concurrently operated as light emitters while four other transceivers are concurrently operated as light detectors. The distance between a light emitter and a light detector is equal to 2 L. In this case, it is possible to investigate two channels each corresponding to an emitter-detector distance equal to (2√2)*L.

As illustrated in FIG. 9a-9d, by dynamically changing the pattern configuration of active transceivers, it is possible to change the tissue region and/or the tissue depth to be investigated. In one embodiment, it is possible to adjust the measurement sensitivity and/or improve the Signal to Noise Ratio (SNR) by dynamically changing the distance between emitters and detectors while not changing their physical positions.

In one embodiment, the probes, i.e. the at least one light emitter, the at least one light detector, and the at least one transceiver, are directly and removably secured to the subject at an adequate location for sensing the tissue to be investigated.

In another embodiment, the probes may be fixedly or removably secured to a frame on which the subject has to abut a part of his body for sensing a tissue.

In a further embodiment, the probes are fixedly or removably secured to a support or frame that is removably securable to the subject. In another embodiment, the probes are remotely and optically connected to a support that is be removably secured to the subject.

FIG. 10a illustrates one embodiment of a support comprising an array of transceivers that may be operated as described above. Each transceiver comprises a light emitter such as a laser diode, a light emitting diode, or the like, and a light detector such a PIN photodiode for example. Cooled lead sulfide detectors, cooled photodarlington detectors, photodarlington, cooled germanium detectors, germanium detectors or the like may be used. More specific example comprise: germanium photodiode for wavelengths in the range of 0.8-1.7 microns; indium gallium arsenide (InGaAs) photodiode for wavelengths in the range of 0.7-2.6 microns; indium antimonide (InSb) photoconductive detectors for wavelengths in the range of 1-6.7 microns; indium arsenide (InAs) photovoltaic detectors for wavelengths in the range of 1-3.8 microns; indium antimonide (InSb) photodiode for wavelengths in the range of 1-5.5 microns; lead sulfide (PbS) photoconductive detectors for wavelengths in the range of 1-3.2 microns; lead selenide (PbSe) photoconductive detectors for wavelengths in the range of 1.5-5.2 microns; mercury cadmium telluride (MCT, HgCdTe) photoconductive detectors for wavelengths in the range of 0.8-25 microns; platinum silicide (PtSi) photovoltaic detectors for wavelengths in the range of 1-5 microns.

For each transceiver, the light emitter and the light detector are adjacent together so as to substantially correspond to a same position on the support. In this case, a beam splitter may be used for collecting and injecting light from a same location.

The support may be connected to a remote power source. Alternatively, the support may comprise a battery for powering the other devices included therein.

The support further comprises a communication unit, such as an infrared (IR) or a radio frequency (RF) communication unit for example, for remotely communicating with a control unit and a measurement collecting unit. The control unit can therefore remotely control the transceivers via the communication unit, and the measurement collecting unit can receive the signals indicative of the measured scattered light energy from the transceivers via the communication unit.

FIG. 10b illustrates one embodiment in which the transceivers are not located on the support. In this case, the transceivers are optically connected to the support to be removably secured to the subject via optical waveguides such as optical fibers for example. For example, the support may comprise an array of lenses each optically connected to a respective transceiver by a respective optical fiber. The light emitted by the light emitter of a transceiver propagates in the respective fiber and is focused on the subject by the respective lens. The light detector of a transceiver receives the scattered light collected by the respective lens via the respective optical fiber.

In this case, the light emitter and the light detector of a transceiver may be located at different locations. Beam splitters, optical switches, optical couplers, optical Y-junctions, and the like may be used for optically connecting both the optical emitter and the light detector to a same optical waveguide, such as a same optical fiber, thereby forming a transceiver.

In one embodiment, the supports of FIGS. 10a and 10b can be applied with external pressure. For example, a pad provided with transceivers or fibers is maintained on a subject thanks to an external support that exerts a pressure against the subject. In another embodiment, the supports of FIGS. 10a and 10b may elastic bands (normal or inflatable), rows, straps, strings, or the like, and may work as a harness that are adjusted to muscles, torso, etc. A helmet can also be used. These harnesses or helmets can be made of semi-rigid materials such as reinforced plastics with padded foam, elastomers, polyurethane, or the like.

It should be understood that the above-described probe system may be used to investigate elements present in a subject body and that may diffract light, such as molecules, particles, tracers, or the like. For example, oxygen, mercury, glucose, or the like may be investigated using the above-described probe system. In an example, the above-described probe system may be used to investigate blood cholesterol. In another embodiment, the above-described probe may be used to investigate triglycerides of which high levels in blood have been linked to atherosclerosis, and by extension to heart disease and stroke. In a further example, the above-described probe system may be used to investigate total protein content, for example troponin in skeletal and cardiac muscle, and more specifically troponin T. In patients with stable coronary artery disease, troponin T concentrations have been found to be significantly associated with the incidence of cardiovascular death and heart failure. In still another example, the above-described probe system may be used to investigate creatine kinase (CK) in skeletal muscles, smooth muscles, brain, photoreceptor-cells of the retina, hair cells of the inner ear, spermatozoa, etc. The elevation of CK is an indication of damage to muscle, rhabdomyolysis, myocardial infarction, myositis and myocarditis. In a further example, the above-described probe system may be used to investigate myoglobin in muscle tissues, which is a sensitive marker for muscle injury, making it a potential marker for heart attack.

It should be understood that the wavelength of the light emitted by the transceivers/light emitters is chosen as a function of the elements to be investigated. For example, visible light, infrared light, ultraviolet light, or the like may be used under certain circumstances.

In one embodiment, the above-described probe system can be used in a near infrared spectroscopy (NIRS) system. In this case, measurement of the amount and oxygen content of hemoglobin may be obtained from the scattered light energy measured by the above-described probe system. For example, brain activity of a human subject may be determined using the above-described probe system.

In one embodiment, a first step consists in determining the SNR in order to determine the particular channels and light detectors that return the highest intensity signals. Then, these channels are used for collecting raw data representative of light intensities which are converted into numerical values. These numerical values, corresponding to the tissue light absorption, are processed by a measurement collecting unit provided with an appropriate algorithm, as known in the art, to obtain the topography of the oxygenated and deoxygenated hemoglobin distribution.

The above-described probe system may be used in systems for investigating tissues, such as muscle, breast, tumors, and the like, that can be used to quantify blood flow, blood volume, oxygen consumption, reoxygenation rates and muscle recovery time in muscle, etc.

In one embodiment, the light emitters, such as light emitter 52 for example, and the light emitters of the transceivers are adapted to emit a single wavelength. In another embodiment, the light emitters, such as light emitter 52 for example, and the light emitters of the transceivers are adapted to concurrently emit more than one wavelength.

In one embodiment, the light emitters, such as light emitter 52 for example, and the light emitters of the transceivers are tunable so that the wavelength of the emitted light may be changed. In this case, the wavelength of the emitted light is controlled by the control unit.

In one embodiment, the above-described probe system can integrate a system capable of performing adaptive signal detection. In this case, the gain from the amplifiers and the pattern position are self adjusted to give a higher signal to noise ratio.

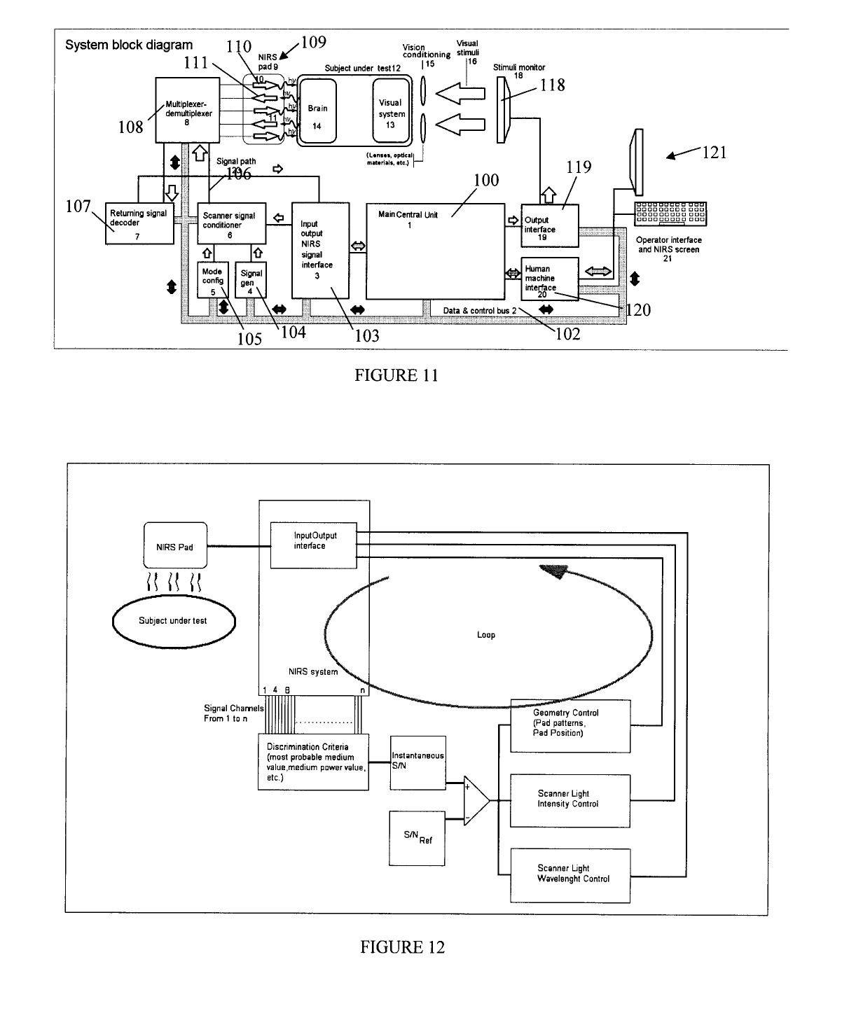

FIG. 11 illustrates one embodiment of a NIRS system comprising the above-described probe system and adapted to measure the cerebral activity of a subject to which visual stimuli are applied. The system of FIG. 11 comprises the following components:

A main central unit 100: this is a processing unit that manages all the other active blocks via the respective buses and the man machine interface. The running programs perform: the system configuration, the evaluation of the raw data trough the NIRS algorithms (finite elements numeric functions determination, etc), the optimization of the data acquisition by modifying the active pattern configuration and by sweeping the brain topography while searching for the best signal detection available (Signal to noise ratio, Stimuli-Signal correlations, etc);

A data and control bus 102 which links all the blocks by transferring all the numeric data, and numeric set up (Gains, attenuations, delay times, synchronism, block multiplexing, etc.);

An input/output NIRS signal interface 103 that manages all the analog and numeric signal data concerning the NIRS itself;

Signal generators 104 that create all the impulses needed for: multiplexing the probes, sweeping the patterns, and the signals sampling;

A mode configurations unit 105 that modifies the scanning control signals according to one of the following operation modes:
manual scanning: the operator defines the probe pattern and its position; and
automatic scanning or close loop operation: the system provides to the subject a programmed stimuli sequence, and defines the best pattern to apply and its position for the best signal detection;

A scanner signal conditioner 106 that manages the signal parameters as light wavelength (for tuned lasers), light intensity, detectors gain, etc;

A returning signal decoder 107 that converts the analog raw data in numeric values;

A multiplexer-demultiplexer 108 that switches each active probe to: configure the patterns, define the pattern sweep, and link the control and data bus with every NIRS Pad probe;

An above-described probe system 109;

An amplifier and a light source; 110 or emitting probe element.

A light detector and an amplifier; 111 or receiving probe element.

A stimuli screen 118 for displaying the visual stimuli;

Outputs interface 119;

A human machine interface manager 120;

An operator interface and NIRS Signals real time screen 121;

Signal paths 123 that link the blocks requiring analog signals. They could be unidirectional, or bi-directional.

FIG. 12 illustrates one embodiment of a system for dynamically and adaptively controlling a probe system such as a NIRS pad used for investigation a subject's brain for example. In this embodiment, the NIRS pad comprises transceivers adapted to measure n channels The system comprises a discrimination unit, an SNR determining unit, a differential amplifier, a geometry control unit, a light intensity control unit, and a wavelength control unit.

The intensity measurement for the n channels is received by the discrimination from an input/output interface that connects the NIRS pad to the other elements of the system. The discrimination unit is adapted to conform the evaluation parameter such as a particular signal channel, the most probable medium value for a combination of signals amongst several channels, the medium power value for the signals of a group of channels, or the like. Statistical analysis such as t-student method, ANOVA method, or the like may be used to help determining which one of the signals or which signals combination is the most representative.

This parameter signal is fed into the SNR determining unit that evaluates its instantaneous SNR. The determined instantaneous SNR is sent to a differential amplifier which compares it to a reference SNR. The differential amplifier outputs an error signal that is fed into the geometry control unit, the light intensity control unit, and the wavelength control unit, which use the received error value as a parameter input for different optimization algorithms.

The geometry control unit is adapted to determine the characteristics of the probe system, e.g. the number of transceivers that are concurrently activated, the relative position of the activated transceivers, the probe pattern geometry, that vary while the error signal is different from zero. Once the error signal is substantially zero, the characteristics for the probe system remain unchanged.

The light intensity control unit is adapted to control the intensity of the light emitted by the transceivers. For example, proportional integrate derivative (PID) may be applied by the light intensity control unit.

The wavelength control unit is adapted to control the wavelength of the light emitted by the transceivers. For example, proportional integrate derivative (PID) may also be applied by the wavelength control unit.

The outputs of the geometry control unit, the light intensity control unit, and the wavelength control unit are transmitted to the NIRS pad via the input/output interface in order to modify the operation of the NIRS pad.

While, in the present example, it is based on SNR, it should be understood that the error signal may be based on other parameters such as the signal mean value, the signal frequency, the signal bandwidth, the signal peak values, or the like.

The embodiments of the invention described above are intended to be exemplary only. The scope of the invention is therefore intended to be limited solely by the scope of the appended claims.

1. A system for optically investigating a tissue of a subject, comprising:
a first probe element, a second probe element, and a third probe element each adapted to be positioned at a respective vertex of a triangle for sensing a given region of a tissue, the first probe element comprising a first light source for emitting light having a first wavelength, the second probe element comprising a second light source for emitting light having a second wavelength and a first photodetector for detecting light having the first wavelength and being scattered by the tissue, and the third probe element comprising a second photodetector for detecting light having the first and second wavelengths and being scattered by the tissue; and
a control unit operatively connected to at least the first and second probe elements, the control unit being adapted to operate the second light source for sensing a first tissue region comprised between the second and third probe elements, and operate the first light source for sensing a second and a third tissue region respectively comprised between the first probe element and the second and third probe elements.

2. The system of claim 1, further comprising a measurement collecting unit connected to the first and second photodetectors for receiving therefrom signals indicative of scattered light energies measured by the first and second photodetectors.

3. The system of claim 1, wherein the control unit is adapted to selectively operate the first and second light sources for selectively sensing the second and third regions, and the first region, respectively.

4. The system of claim 1, wherein the control unit is adapted to concurrently operate the first and second light source.

5. The system of claim 1, wherein the second probe element comprises an optical transceiver.

6. The system of claim 1, wherein the first, second, and third probe elements are each adapted to be positioned at a respective vertex of one of an isosceles triangle, a scalene triangle, and an equilateral triangle.

7. The system of claim 1, wherein the first wavelength emitted by the first light source and the second wavelength emitted by the second light source are substantially identical.

8. The system of claim 1, wherein a distance between the first light source and the first photodetector and a distance between the first light source and the second photodetector are each substantially equal to about three times a tissue depth to be investigated.

9. A method for optically investigating a tissue of a subject, comprising:
positioning, on the subject, a first probe element, a second probe element, and a third probe element each at a respective vertex of a triangle for sensing a given region of the tissue, the at least one first probe element each comprising a first light source for emitting light having a first wavelength, the at least one second probe element each comprising a second light source for emitting light having a second wavelength and a first photodetector for detecting light having the first wavelength and scattered by the tissue, and the at least one third probe element comprising a second photodetector for detecting light having the first and second wavelengths and scattered by the tissue;
operating the second light source for sensing a first tissue region comprised between the second and third probe elements; and
operating the first light source for respectively sensing a second tissue region comprised between the first probe and the second probe, and a third tissue region comprised between the first probe and the third probe.

10. The method of claim 9, wherein said operating the first light source and said operating the second light source are selectively performed for selectively sensing the second and third regions, and the first region, respectively.

11. The method of claim 9, wherein said operating the first light source and said operating the second light source are performed substantially concurrently.

12. The method of claim 9, wherein the second probe element comprises an optical transceiver.

13. The method of claim 9, wherein said positioning comprises positioning the first probe element, the second probe element, and the third probe element each at a respective vertex of one of an isosceles triangle scalene triangle, and an equilateral triangle.

14. The method of claim 9, wherein the first wavelength emitted by the first light source and the second wavelength emitted by the second light source are substantially identical.

15. The method of claim 9, wherein said positioning comprises positioning the first, second, and third probe elements so that a distance between the first light source and the first photodetector and a distance between the first light source and the second photodetector are each substantially equal to about three times a tissue depth to be investigated.

16. A probe device for optically investigating a tissue of a subject, comprising:
a first probe element, a second probe element, and a third probe element each to be positioned at a respective vertex of a triangle for sensing the tissue, the first probe element each comprising a first light source for emitting light having a first wavelength, the second probe element each comprising a second light source for emitting light having a second wavelength and a first photodetector for detecting light having the first wavelength and scattered by the tissue, and the third probe element comprising a second photodetector for detecting light having the first and second wavelengths and scattered by the tissue, the second light source and the first photodetector being respectively adapted for sensing a first tissue region comprised between the second and third probes, and a second and third tissue regions comprised between the first probe and the second and third probes.

17. The probe device of claim 16, wherein the first, second, and third probe elements are each removably securable to the subject at an adequate location for sensing the tissue.

18. The probe device of claim 16, further comprising a frame securable to the subject.

19. The probe device of claim 18, wherein the first, second, and third probe elements are secured to the frame.

20. The probe device of claim 18, wherein the first, second, and third probe elements are remotely and optically connected to the frame.
